(19) United States
(12) Patent Application Publication
(10) Pub. No.: US 2025/0003944 A1
(43) Pub. Date: Jan. 2, 2025

(54) METHOD FOR THE QUANTITATIVE MEASUREMENT OF AN ELEMENT IN A PIECE

(71) Applicant: SAFRAN HELICOPTER ENGINES, BORDES (FR)

(72) Inventors: Cyril Roger VERNAULT, MOISSY-CRAMAYEL (FR); Lucie ANSSEMS, MOISSY-CRAMAYEL (FR); Erika Michèle Mariane BESOMBES, MOISSY-CRAMAYEL (FR); Manon Paule Jeanne GAULHET, MOISSY-CRAMAYEL (FR)

(21) Appl. No.: 18/700,575

(22) PCT Filed: Oct. 10, 2022
(86) PCT No.: PCT/FR2022/051903

§ 371 Date: Apr. 11, 2024

(30) Foreign Application Priority Data
Oct. 12, 2021 (FR) FR2110757

(51) Int. Cl. G01N 33/204; G01N 1/28; G01N 23/20; G06T 7/00

(52) U.S. Cl. CPC G01N 33/204; G01N 1/286; G06T 7/0004; G01N 2001/2873; G01N 23/20; G06T 2207/10056; G06T 2207/30136

(57) ABSTRACT

A method for the quantitative measurement of an element in a metal test specimen which has received a thermochemical surface treatment, the method including—a—making a cut in the test specimen;—b—taking a photograph, using an optical microscope, of the cut on a surface S with magnification;—c—calculating the ratio of the surfaces s occupied by the element with respect to the surface S in the photograph by an image-processing tool; and—d—obtaining the content of the element in the surface S of the test specimen. A profile of the content of undesirable elements can thus be obtained without the subjective aspect of human checking, which profile is representative of a content obtained by acquiring diffraction of X-rays produced using a synchrotron.

### TECHNICAL FIELD OF THE INVENTION

The technical field of the invention is that of the measurement of the content of a specific element in a piece and more particularly how to determine whether the piece has an acceptable content or not.

### TECHNOLOGICAL BACKGROUND TO THE INVENTION

In order to control quality of pieces which have received a thermochemical surface treatment aimed at hardening surface layers, such as carburising, the inspection after these operations on power transmission pieces is particularly critical, especially for those fitted to helicopter turbine engines.

The performance of these pieces lies in the control of the mechanical properties obtained by these treatments, in particular the thermochemical carburising treatments of the pinions, as well as the absence, during engine operation, of any alteration in the dimensions of the loaded surfaces.

For this reason, it is essential to ensure that the microstructure of the surface layers hardened by these treatments does not contain undesirable residual elements, such as austenite, with a content different from that required by the applicable specification.

Indeed, this metallurgical phase which may result from the thermochemical surface treatment operation may:
locally alter mechanical properties of the treated layers
lead to microstructural instability which, under the action of mechanical loads and temperatures encountered during operation, can lead to other phases by crystallographic transformation and induce a possible impact on the integrity of the pieces.

Consequently, during inspections after the thermochemical surface treatment operations provided for in the production range of series pieces, it is required to check that the content of undesirable residual elements, such as austenite, does not exceed the specification in force. For example, for austenite, this maximum value is set to 15%.

Today, the measurement of the content of an undesirable residual element such as austenite in a steel uses the X-ray diffraction method. This offers the possibility of obtaining a quantitative result. It is based on the use of a diffraction diagram and the measurement of the intensity of the diffraction lines associated with the crystallographic phases present.

However, its implementation is most often limited to carrying out an analysis from the surface of the piece/sample studied. Thus, the zone actually analysed is located in the first layers of the material. In the case of gear pieces, however, the interest of this inspection is very limited insofar as their working surfaces are in most cases reworked by machining after the thermochemical surface treatment. To obtain results deeper into the material, it is necessary to make a profile of the content of undesirable elements by carrying out X-ray diffraction analyses following incremental removal of material by electrolytic polishing. The drawback of this approach is that it is only available in a laboratory environment and is very time-consuming. It is therefore difficult to apply industrially in a production flow.

For these reasons, in an industrial environment this inspection is carried out using metallographic observations made from micrographic sections perpendicular to the surface of the pieces/sample in the direction of the carbon enrichment gradient. The qualified inspectors in charge of these analyses make their judgements by comparison with standard photographs illustrating different levels of content of these residual elements. Their assessment obtained by comparison is therefore “pseudo-quantitative” rather than being an actual quantitative measurement.

Although this method has the advantage of being easy to implement, the results obtained depend significantly on the acuity of the inspector (even if he/she has received adequate training, there will be an inherent variability), on the bank of standard images which may have been built up by the manufacturer through his/her experience in his/her field of product application, as well as on the correspondence established with the X-ray diffraction method.

This situation poses a problem when the content observed gets close to the acceptable limits and, in order to make a decision, it is necessary to use the so-called reference X-ray diffraction method. This involves sending a beam of X-rays onto the surface of the piece/sample, which diffracts according to Bragg's law onto the crystallographic planes of the phases present in the material. Using the diffraction peaks of the X-rays (angular positions and intensities of the peaks) makes it possible to identify and quantify the phases. However, due to its principle, this method is limited to a depth of around ten microns, corresponding to the penetration of the X-rays and their interaction with the crystallographic planes of the material (irradiated zone).

The volume of material thus studied therefore concerns the first surface layers. These conditions are not equivalent to microscopic observation of a micrographic section which covers a depth in relation to the surface of the control piece of up to a few millimetres or more if necessary. The result thus obtained by X-ray diffraction corresponds to that which would be obtained by analysing a narrow band of the image made on the micrographic section.

There is therefore no inspection method that can obtain a result of equivalent quality to that obtained by X-ray diffraction, over a sufficient depth that can be used in an industrial environment.

### SUMMARY OF THE INVENTION

The invention offers a solution to the problems previously discussed, by making it possible to measure an undesirable element in a metal piece analytically and quantitatively. By virtue of the invention, a method of analysis is available in which the result of the content of the undesirable element is a value measured and obtained completely analytically and quantitatively; it concerns the zones of interest in the piece/sample giving the associated values of the content sought, can be correlated with the so-called X-ray diffraction method and can be obtained easily in an industrial environment.

The invention relates to a method for quantitatively measuring an element in a metal control piece which has received a thermochemical surface treatment, characterised in that it comprises the following steps of:
a—making a section in the control piece,
b—taking a photograph by an optical microscope of said section on a surface S with magnification,
c—calculating the ratio of the surfaces s occupied by the element to the surface S on the photograph using an image processing tool,
d—obtaining the content of the element in the surface S of the control piece.

It is thus possible to obtain a profile of the content of undesirable elements (s/S) without the subjective aspect or variability of inspection by a human and which is representative of a content obtained by acquisition of the X-rays from X-ray diffraction using a synchrotron. For example, Stream Essential™ software or ImageJ™ software may be used.

Advantageously, the image processing tool measures the number of pixels occupied by the element in the surface S. The measurement is thus particularly accurate.

Advantageously, the section is marked by aligned equidistant points, known as micro-hardness points. This alignment provides a reference mark for the entire depth of the thermochemical surface treatment.

Advantageously, the points are preferably spaced apart by a distance equal to the side of the surface S, for example 80 μm. These points serve as a reference mark for location and their spacing is adapted to the irradiated surface, to have a continuous view of the depth of the thermochemical surface treatment.

Advantageously, steps b, c and d are repeated on other surfaces Sn disposed in a line and side by side. This repetition makes it possible to cover a larger surface and to have a view of the entire depth of the thermochemical surface treatment. Said surfaces are positioned by virtue of the above reference points.

Advantageously, the surface S is 80 μm×80 μm. This surface dimension has been limited to correspond to the size of the X-ray beam, which has a section area of 80×80 μm², because this dimension offers the advantage of having a relatively constant content of undesirable elements; the gradient of the thermochemical surface treatment being perceptible only for a more substantial extent.

Advantageously, the undesirable element is austenite. During thermochemical surface treatment operations by carburising, the presence of residual austenite in the carburised layers of steel pieces and in particular in the 16NiCrMo13 alloy is linked to carbon enrichment during carburising. Austenite locally alters the mechanical properties of the carburised layers and its microstructural instability, which under the action of mechanical loads and temperatures encountered during operation, can lead by crystallographic transformation to other phases and thus induce dimensional deformations of the surfaces of the piece. Carbon enrichment of the piece by diffusion from the surface during the carburising operation results in promoting the stability of the austenite phase which remains present after returning to room temperature.

Advantageously, the control piece comprises a notch. This notch makes it possible to simulate a toothing, it represents the replica of two toothing flanks of a pinion and makes it possible to reproduce with similarity the diffusion phenomenon for this geometry.

According to one particular characteristic, the production piece, which has undergone the same treatment as the control piece, is declared to be compliant if the content of the element in the section of the control piece is less than a limit content and not in conformity if the content of the element is greater than the limit content. For example, for austenite, the content should be less than 15%.

### BRIEF DESCRIPTION OF THE FIGURES

The figures are set forth by way of indicating and in no way limiting purposes of the invention.

FIG. 1 shows the different analysis zones according to the methods used, between the conventional X-ray diffraction method and the micrographic section method,

FIG. 2 is a cross-section view of a control piece according to the invention;

FIG. 3 is a detail of part of the control piece with points serving as reference marks;

FIG. 4 shows an image obtained by synchrotron-implemented X-ray from the micrographic section of the control part;

FIG. 5 shows the image of FIG. 4 after processing by image processing software;

FIG. 6 shows a comparison of the residual austenite content profiles obtained from the X-ray diffraction analysis methods and the inventive method.

### DETAILED DESCRIPTION

The figures are set forth by way of indicating and in no way limiting purposes of the invention.

Unless otherwise specified, a same element appearing in different figures has a unique reference.

In the remainder of the description, the example used concerns a thermochemical surface treatment such as carburising, but other types of thermochemical surface treatment may be contemplated, such as carbonitriding, or carburising-nitriding sequence, etc . . . , The undesirable element here will therefore be austenite, but other elements can be measured.

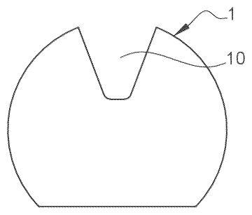

It can be seen in FIG. 1:
a piece 1 on which a thermochemical surface treatment 4 has been performed,
the volume 2 analysed by conventional synchrotron X-ray diffraction, which covers only part of the thermochemical surface treatment 4, and
a surface 3 which is the subject of micrographic analysis, following cutting of the piece 1, and which only covers this surface. These two techniques therefore give only fragmentary results.

For carburising, carbon enrichment of the treated piece is carried out by diffusion of the carbon from the surface. This diffusion thus creates a carbon content profile which decreases from the surface of the piece towards its core along the arrow 40. This carburising is carried out in a furnace in which a control piece is placed which will therefore receive the same treatment as the production piece to be analysed.

This control piece 1, illustrated in FIG. 2, is substantially cylindrical and has a notch 10, in this case this notch 10 represents a replica of a pinion toothing flank (engine piece usually carburised) and enables the diffusion phenomenon to be reproduced with similarity for this geometry. Carbon enrichment takes place simultaneously on different surfaces of the control piece, especially on the “two sides” for each tooth crest.

The control piece 1 undergoes a carburising operation with a deliberately very high carbon enrichment (content>0.95% in the first tenth of the depth of the control piece) thus enabling an increase in residual austenite to be obtained from the surface to the depth of the carburised layer. A slice in the order of 1 mm thick is taken from this control piece.

The control piece is subjected to a preparation consisting in:
a standard micrographic preparation, in order to obtain a poly mirror (these steps have been frozen in order to ensure repeatability):
making a micrographic section by cutting a slice perpendicular to the carbon gradient in the control part;
coating the slice with resin,
polishing with abrasive paper (grades 120 and 600), then diamond spray (9 and 3 μm).
A micro-hardness point 12 filiation from the surface at the top of the slice to a depth of more than 1.28 mm, with a distance between indentations of 80 μm. This operation provides visual reference points on the slice.
etching with a nitric acid solution, such as 5% Nital at room temperature, with an etching time of between 3 and 30 sec, to reveal the microstructure and in particular the residual austenite, with a view to its observation and quantification.

This slice is studied by synchrotron-implemented X-ray diffraction, which enables local volume analyses to be carried out as the X-rays penetrate the material. The effective analysis zone therefore corresponds to a volume associated with the reduced and selected irradiated zone. This method thus makes it possible to obtain a profile of the residual austenite content by studying successive fields made along a virtual line identified by points 12 corresponding to the carbon diffusion gradient. In this example, the successive fields are made every 80 μm from the surface of the slice of the control piece to the depth of 1.28 mm, for example. In this example, the surface dimension of the fields has been limited by fixing the beam size at a section area of 80×80 μm². This size also offers the advantage of having a relatively constant residual austenite content; the increase in austenite content being perceptible only for a more substantial extent.

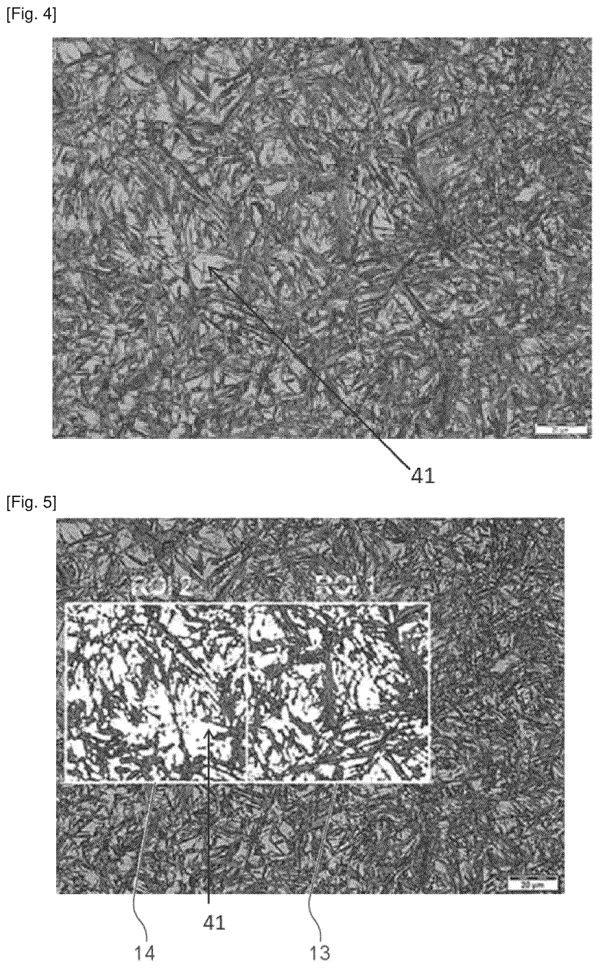

The metallographic analyses are carried out using an optical microscope with adapted magnification allowing the observation of a surface of identical size to the irradiated surface for each zone of material volume studied using a synchrotron, that is 80×80 μm² here. Digital photographic pictures (see FIG. 4) corresponding to each zone studied by X-ray diffraction are obtained with the aid of the indentations of micro-hardness points 12 used as reference marks on the micrographic section.

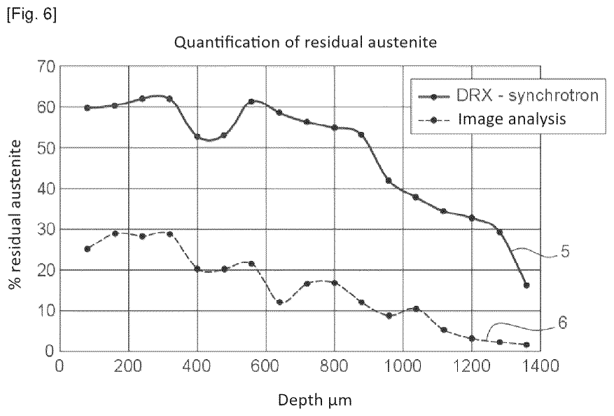

From these photographs, quantification of the residual austenite content can be measured using an image processing and analysis tool. By calculating the ratio of the sum of the surfaces occupied to the total surface of the zone analysed, the residual austenite content can be obtained. FIG. 5 shows a view of two different zones 13 and 14 of the slice, corresponding to two contiguous sectors along the direction 40 of carbon diffusion and marked by virtue of the filiations in FIG. 3. It can be seen in FIG. 4 the raw images where the element 41 is residual austenite, shown in grey, and in FIG. 5 the images after processing by the software where the phase of the identified element 41, in this case residual austenite, appears as white pixels.

In order to validate these measurements, a comparison has been carried out to validate their correlation with the measurements made by X-ray diffraction and those made on the surface of a section. Measurements made at different depths are visible in FIG. 6. The curve 6 shows the residual austenite content obtained by the method according to the invention, while the curve 5 is that obtained by the synchrotron X-ray diffraction method. It can be noticed that the two curves are substantially parallel, showing that there is indeed a correlation between these two measurement methods, the deviation coming from the fact that the measurement according to the invention is made on a surface (surface measurement) whereas the X-ray measurement integrates a thickness (volume measurement). The measurement according to the invention is therefore representative of the residual element content in the depth of the thermochemical surface treatment. The invention can therefore be applied to the inspection for approval of the thermochemical treatment, on a production monitoring control piece (as represented in FIG. 2) but also on a real power transmission piece, or any other carburised material sample and in all the zones considered to be of interest in these pieces.

1. A method for quantitatively measuring an element in a metal control piece which has received a thermochemical surface treatment, the method comprising the following steps of:
a—making a section in the control piece,
b—taking a photograph by an optical microscope of said section on a surface S with magnification,
c—calculating a ratio of surfaces s occupied by the element to the surface S on the photograph by an image processing tool,
d—obtaining the content of the element in the surface S of the control piece.

2. The method according to claim 1, wherein the image processing tool measures the number of pixels occupied by the element in the surface S.

3. The method according to claim 1, wherein the section is marked by aligned equidistant points.

4. The method according to claim 3, wherein the points are spaced apart by a distance equal to the side of the surface S.

5. The method according to claim 1, wherein steps b, c and d are repeated on other surfaces Sn disposed in a line and side by side.

6. The method according to claim 1, wherein the surface S is 80 μm×80 μm.

7. The method according to claim 1, wherein the element is austenite.

8. The method according to claim 1, wherein the control piece comprises a notch.

9. The method according to claim 1, wherein a production piece which has undergone a same treatment as the control piece is declared to be compliant if the content of the element in the section of the control piece is less than a limit content and not in conformity if the content of the element is greater than the limit content.
